(19) United States
(12) Patent Application Publication
(10) Pub. No.: US 2009/0157186 A1
(43) Pub. Date: Jun. 18, 2009

(54) INTER-VERTEBRAL IMPLANT

(75) Inventor: Friedrich Magerl, St. Gallen (CH)

Correspondence Address: BROWDY AND NEIMARK, P.L.L.C.;624 NINTH STREET, NW, SUITE 300, WASHINGTON, DC, 20001-5303, US

(73) Assignee: SEPITEC FOUNDATION, Vaduz (LI)

(21) Appl. No.: 12/063,552

(22) PCT Filed: Aug. 3, 2006
(86) PCT No.: PCT/CH2006/000404

§ 371 Date: Sep. 30, 2008

(30) Foreign Application Priority Data
Aug. 11, 2005 (EP) 05405472.1

(51) Int. Cl. A61F 2/44

(52) U.S. Cl. 623/17.16; 623/17.11

(57) ABSTRACT

The inter-vertebral implant comprises a first part (2, 21, 26) and a second part (3, 22, 27), which may be uniportally implanted in a disc cavity (B) and connected to each other therein. The parts (2, 3; 21, 22; 26, 27) comprise an opening (4, 5; 28, 29) for housing spongiosa or bone replacement material. Both parts (2, 3; 21, 22; 26, 27) have connector means (8, 9; 23, 24; 31, 35), which engage with each other on implantation in the disc cavity (B) and thus connect both parts (2, 3; 21, 22; 26, 27) to each other. At least one part (3, 22) preferably comprises a groove (8, 23) in which a spring (9, 24) of the other part may be introduced in a sliding manner. The groove (8, 23) and the spring (9, 24) are arranged on the inner narrow faces (10, 11) of both parts (2, 3; 21, 22) which are to come together. The implant may be implanted uniportally through a transforaminal or an extraforaminal opening (T, E) equally carefully.

The invention relates to an intervertebral implant for connecting adjacent vertebrae, with a first part and a second part, which can be inserted through an opening (uniportally) into an intervertebral disk cavity and can be connected to one another therein, and which in each case have a central cavity for housing spongiosa or bone replacement material.

Intervertebral implants have been known for some time now. Intervertebral implants made up of two segments or parts are also known. With such a comparatively large implant it is possible to support the vertebrae more widely than would be possible with a smaller implant. With such wider support the vertebra can less easily tip over the implant. Also, the surface pressure exerted by the implant on the end plates of the vertebrae is less than in a narrower implant. Finally, such an implant is less dependent on the configuration of the end plates of the vertebrae.

An intervertebral implant of two parts which can be connected to one another in situ is known from WO 03/071992. Both parts are designed as L-shaped cages and can in each case accommodate spongiosa or bone replacement material in a recess. Both parts must be inserted using a special instrument through two openings (biportally) from behind (posterior) into the intervertebral disk cavity. Two avenues of access must accordingly be made to the intervertebral disk cavity and the disk, to left and right, constituting major destruction to the dorsal vertebral elements as well as traumatizing of the spinal canal.

U.S. Pat. No. 5,861,041 discloses an intervertebral implant which likewise comprises two parts or respectively segments. In the side elevation the implant is wedge-shaped and in plan view it is somewhat elliptical. Both parts are guided successively and uniportally into the intervertebral disk cavity and lie behind one another in a sagittal direction. So that both parts cannot be detached from one another they are screwed together after implanting. Both parts can be inserted via a rear access uniportally and finally rest transversally in the intervertebral disk cavity. They can however also be inset via transforaminal access, though then they must be rotated into a transverse position.

The object of the invention is to provide an intervertebral implant of the known type, which can be inserted into the intervertebral disk cavity even more easily and securely uniportally from behind.

This task is solved with an inventive intervertebral implant by both abovementioned parts having connecting means, which engage in one another as the second part is being introduced to the intervertebral disk cavity, thus interconnecting both parts. In the case of the inventive intervertebral implant a first part and a second part are thus provided, which can be introduced into the intervertebral disk cavity uniportally transforaminally or extraforaminally. As the second part is inserted into the intervertebral disk cavity the second part is connected in situ to the first part detachably or undetachably. Joining or respectively connecting both parts in situ results overall in a broad implant having the abovementioned advantages. The spongiosa or respectively bone replacement material can already be filled in the abovementioned parts prior to insertion. Both parts can be designed wedge-shaped, so that the intervertebral disk cavity can be widened with them, promoting stable clamping of the intervertebral implant and thus stability for spondylolysis. Prior to both parts being inserted the intervertebral disk cavity is spread apart using distracters, known per se. The final distraction preferably occurs via both parts or respectively the intervertebral implant itself.

According to a further development of the invention it is provided that at least one part has a groove, in which a tongue of the other part can be inserted in a sliding manner. In the process, both parts can be joined as they are implanted and thus aligned to one another and stabilised. The groove and the tongue are preferably arranged on inner narrow sides of both parts which are to come together. As the second part is being implanted it is guided on the already implanted first part and can thus be brought into the desired position better than previously.

According to a further development of the invention it is provided that the groove and the tongue form a dovetail joint. This results in both parts not being able to be detached from one another in a sagittal direction. This results in a particularly stable connection between both parts. Despite this, both parts can be manufactured easily and robustly.

Both parts of the intervertebral implant are preferably annular, in particular oval rings, and can also be configured wedge-shaped in the transversal axis with respect to lordosis to varying extent. Both parts can be designed open or also one part open and one closed.

Both parts can preferably be inserted into the intervertebral disk cavity such that they are arranged behind one another in a sagittal direction. When both parts are inserted they rotate by themselves from the direction of insertion to the end position, if, according to a further development of the invention, they have guide means and in particular a guide notch, a structured surface and/or sharp edges.

According to a further development of the invention it is provided that both parts can be locked together. This prevents both parts from sliding past one another.

The locking is preferably configured such that locking is completed automatically in situ.

Further advantageous characteristics will emerge from the dependent claims, the following description and the diagram.

Embodiments of the invention are explained hereinbelow in greater detail by means of the diagram, in which:

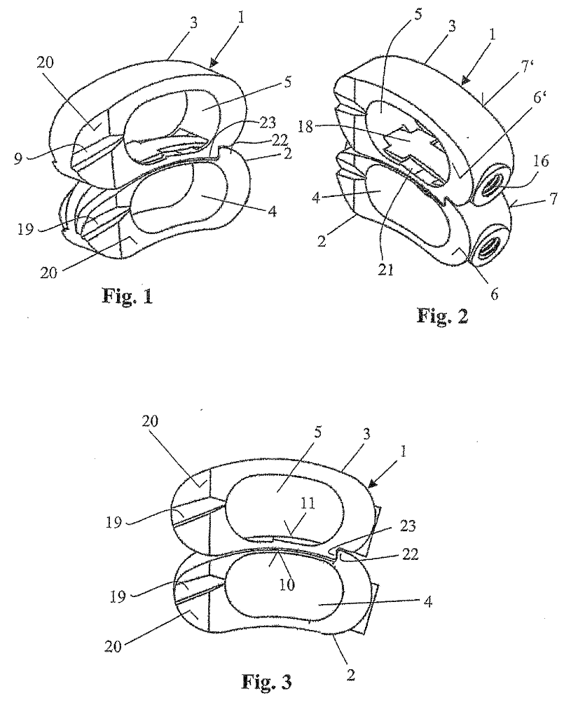

FIG. 1 illustrates a spatial schematic view of an inventive implant,

FIG. 2 illustrates another spatial view of the implant according to FIG. 1,

FIG. 3 illustrates a side elevation of the implant,

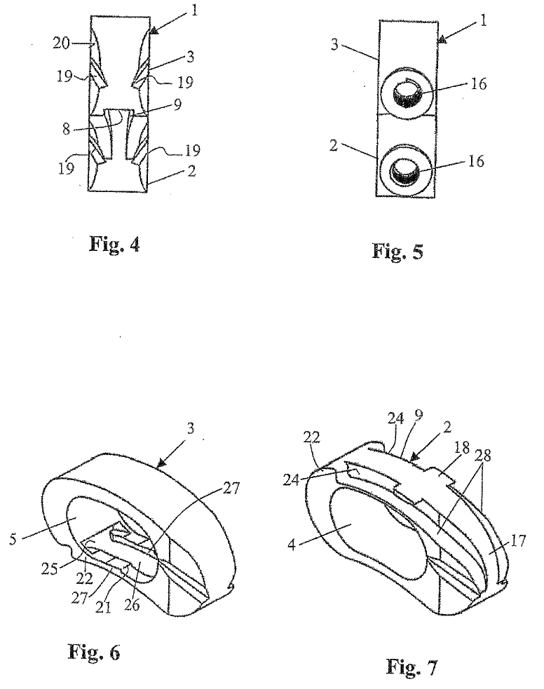

FIG. 4 illustrates another view of the implant,

FIG. 5 illustrates a spatial view of the implant,

FIG. 6 illustrates a spatial view of the other part of the implant,

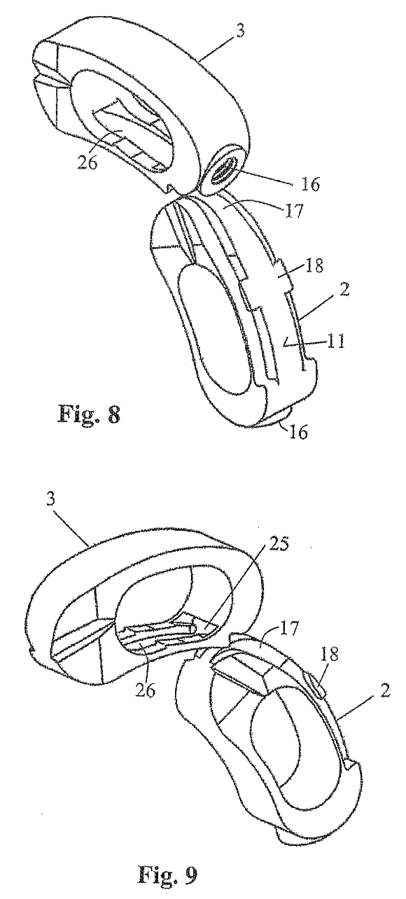

FIG. 7 illustrates a view of one part of the implant,

FIGS. 8 to 11 schematically illustrate individual phases during connecting of both parts of the implant, and

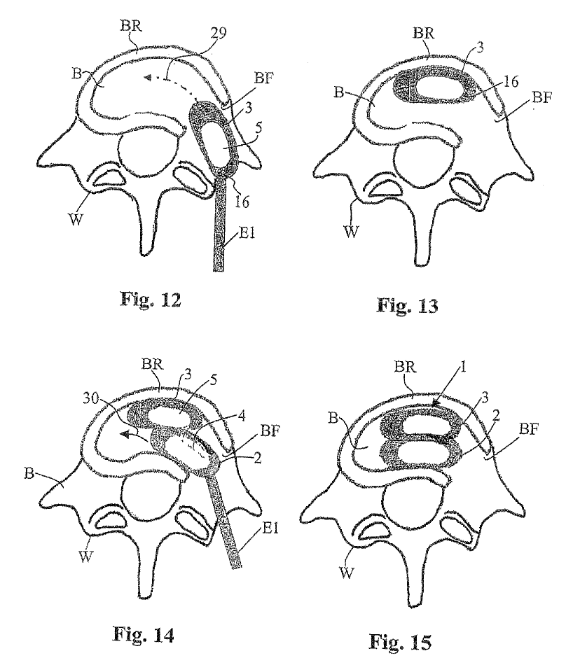

FIGS. 12 to 15 schematically illustrate individual steps during introduction and connecting of both parts in the intervertebral disk cavity.

The implant 1 shown in FIGS. 1, 2 and 3 comprises two parts 2 and 3, which in each case in the view according to FIG. 3 are designed bean-shaped or respectively annularly, and which have a continuous cavity 4 or respectively 5 open on two sides for housing spongiosa or bone replacement material, not shown here. Both parts 2 and 3 are in each case preferably made monobloc from a suitable biocompatible material, for example titanium or a reinforced plastic, for example carbon-fibre-reinforced PEEK. The part 2, which is the front part in the implanted state according to FIG. 15, has a narrow side 11, lying at least in part on a narrow side 10 of the part 3. The narrow side 11 is convex and the narrow side 10 is configured concave. Both parts 2 and 3 also have surfaces 6 and 7 or respectively 6′ and 7′; the surfaces 6 and 6′ and the side faces 7 and 7′ lie substantially in the same plane. The faces 6, 6′, 7 and 7′ in each case have at one and of the part 2 or respectively of the part 3 a distraction surface, by which the corresponding end is formed wedge-shaped. These wedge-shaped bevels enable the vertebrae to be pushed apart to increase the intervertebral distance. In addition, guide grooves 19 are arranged in the region of these distraction surfaces 20, which form depressions and which in each case guide both parts 2 and 3 into the intervertebral disk cavity when being inserted. Reference is made to patent application PCT/EP 2004/002466 of the applicant.

The part 2 has on the narrow side 11 a tongue 9, extending substantially over the entire narrow side 11. The tongue 9 has surfaces 24 which run parallel to each other, with the exception of in the vicinity of an insertion part 17. In the insertion part 17 these surfaces 24 converge wedge-shaped. In the position shown in FIG. 7 a shoulder 22, wider than the tongue 9, is arranged at the left end of the tongue 9. The shoulder 22 forms a stop for the part 3, as explained in greater detail hereinbelow. Formed between the shoulder 22 and the insertion part 17 on the tongue 9 is a locking part 18, which is designed like a plate or a wing, as is evident, and which protrudes over the surfaces 24 to both sides.

On the face 10 the part 3 has an insertion opening 25, bordered by a continuous slot 26, according to FIG. 6, which finally transitions into a groove 8, according to FIG. 4. Formed in the region of the opening 25 is a shoulder 22, which cooperates with the abovementioned shoulder 23. Running laterally near the slot 26 are bridges 27, on which a step 21 is formed on the inside in each case, cooperating with the locking part 18 and forming a locking organ with the latter. The slot 26 and the opening 25 are designed such that the tongue 9 can be inserted into them. At the time of insertion, glide surfaces 28, which are arranged near the tongue 9 according to FIG. 7, form contact surfaces on the narrow side 10 in the region of both bridges 27. The bridges 27 are substantially approximately the same width as the glide surfaces 28. The curvature of the glide surfaces 28 is configured to correspond to the curvature of the narrow side 10, so that flat contact results on sliding open.

The surfaces 24 are not compulsorily parallel to one another, but can also be inclined such that the tongue 9 is trapezoid in cross-section. The slot 26 is designed correspondingly. In this case the result is a dovetail configuration.

Parts 2 and 3 in each case have a screw hole 16, to which the former can be connected for implanting using an insertion instrument, not shown here. Such insertion instruments are known to the expert. Reference is again made to the abovementioned PCT/EP 2004/002466.

Both parts 2 and 3 can be joined together in situ in the intervertebral disk cavity B to form the implant 1 shown in FIGS. 1 to 3 and connected firmly to one another. At this, firstly the part 2 or respectively the front part and then the part 3 or respectively the rear part is inserted, as shown in FIGS. 12 to 15.

The part 3 is guided on the implanted part 2 and inevitably connects to the latter. The production of this connection is explained in greater detail hereinbelow by means of FIGS. 6 to 11.

FIGS. 6 and 7 show both parts 2 and 3 prior to implanting. The parts 2 and 3 are implanted through an opening BF in the intervertebral disk cavity B and thus uniportally and preferably laterally, as shown in FIGS. 12 to 15.

Connecting both parts 2 and 3 to form the implant 1 is explained in greater detail hereinbelow by means of FIGS. 8 to 11. This joining occurs in situ in the intervertebral disk cavity B, as explained above.

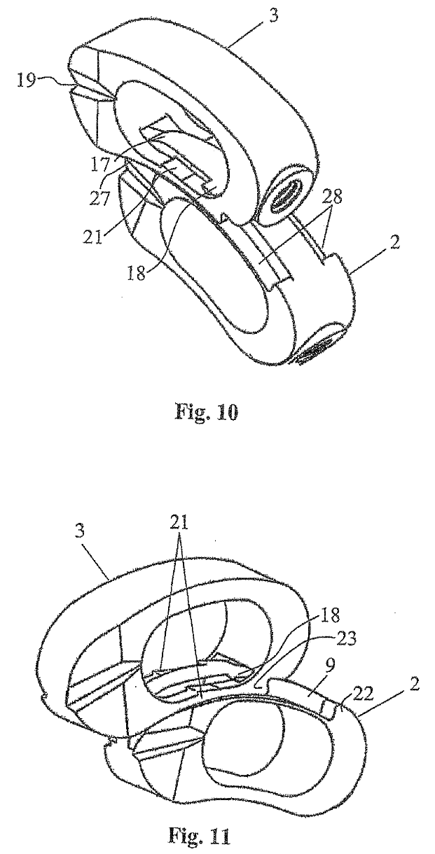

In the arrangement according to FIG. 3 it is assumed that the part 3 is inserted into the intervertebral disk cavity B with an insertion instrument, not shown here. The part 2 is inserted through the same opening BF likewise into the intervertebral disk cavity B, using the same instruments. As FIG. 9 shows, the part 2 is now guided such that the forward tapering wedge-shaped insertion part 17 can be introduced through the opening 25 into the slot 26. FIG. 10 shows the state in which this insertion part 17 is already in the slot 26. As is evident, the part 2 with the glide surfaces 28 is guided on the part 3. Other guide means are the tongue 9 with the insertion part 17, which glide along the bridges 27. In the state shown in FIG. 10 the locking part 18 is also inserted through the opening 25 into the part 3 and now lies on top on the bridges 27. The part 2 is now pushed further in the same direction, whereby it is guided on the part in a sliding manner, as explained. The part 2 now reaches the final position shown in FIGS. 1 to 3 via the position shown in FIG. 11. The locking part 18 is pushed over the ramp-like rising steps 21 and jumps over them finally, locking the part 2 with the part 3. This locking can be detachable or also undetachable. The end position is determined by a stop of the shoulders 22 on the shoulders 23. The part 2 is thus locked with the part 3 and these parts thus form the implant 1.

The locking between both parts 2 and 3 can also occur using other appropriate catch means or the like. Such catching or respectively locking is however not mandatory. A design is also conceivable in which the two parts 2 and 3 are not locked. In the case of such a design the groove 8 and the slot 46 are preferably configured as a type of dovetail groove, in which the tongue 9 with corresponding cross-section is set. This results in a connection which firmly joins both parts 2 and 3 together in a sagittal direction.

FIGS. 12 to 15 schematically illustrate insertion and connection of the two parts 2 and 3 through the opening BF to form the implant 1 in the intervertebral disk cavity B.

FIG. 12 shows the part 3, which is inserted into the cleaned-out intervertebral disk cavity B, using an insertion instrument El in the direction of the arrow 29. The abovementioned distraction takes place with insertion. Also, as likewise mentioned earlier, the part 3 is guided in the intervertebral disk cavity B. The guide elements are in particular the guide groove 19 and sharp edges and surface structures according to PCT/EP2004/002466. Finally, as shown in FIG. 13, the part 3 lies between both vertebrae W, (whereof only one of the vertebrae is shown here) in the intervertebral disk cavity B in the position provided. Here, the part 3 is aligned with respect to the intervertebral disk cavity B as shown and with its convex narrow side lies on the disk rings BR of both adjacent vertebrae W.

As shown in FIG. 14, the part 2 is now inserted into the intervertebral disk cavity B with an insertion instrument El. The part 3 remains in the position shown in FIG. 13. As mentioned earlier, the part 2 is guided on the part 3 and is pushed further forward in the direction of the arrow 30. During this movement the abovementioned catch connection is formed. The resulting implant 1 shown in FIG. 15 thus forms a unit. As already mentioned, both parts 2 and 3 can also be connected in another way, whereby locking is not compulsory. A design in which the engaging occurs via a groove and a tongue only is also conceivable. The groove can be a dovetail groove or a standard groove with parallel side walls.

### LIST OF REFERENCE DESIGNATIONS

1 implant
2 part
3 part
4 cavity
5 cavity
6 surface
7 surface
8 groove
9 tongue
10 narrow side
11 narrow side
16 screw hole
17 insertion part
18 locking part
19 guide groove
20 distraction surface
21 step
22 shoulder
23 shoulder
24 surface
25 opening
26 slot
27 bridges
28 glide surfaces
29 arrow
30 arrow
B intervertebral disk cavity
BF disk opening (window)
BR disk ring
El insertion instrument

1. An intervertebral implant for connecting adjacent vertebrae, with a first part and a second part, which can be inserted through an opening into an intervertebral disk cavity and can be connected to one another therein, and which in each case have a central cavity for housing spongiosa or bone replacement material, wherein both said parts have connecting means, which engage in one another as the second part is being introduced to the intervertebral disk cavity, thus interconnecting both parts.

2. The implant as claimed in claim 1, wherein at least one part has a groove or a slot, in which a tongue of the other part can be inserted in a sliding manner.

3. The implant as claimed in claim 2, wherein the tongue is arranged on a convex narrow side of the corresponding part.

4. The implant as claimed in claim 2, wherein both parts can be connected with a dovetail joint.

5. The implant as claimed in claim 1, wherein the first part and the second part have connecting means, with which both parts can be latched or locked on to one another.

6. The implant as claimed in claim 5, wherein a part has a catch or locking part as connecting means, which can be inserted through an opening of the other part into the latter.

7. The implant as claimed in claim 1, wherein both parts are designed bean-shaped.

8. The implant as claimed in claim 1, wherein one part has two bridges arranged at a distance from one another, between which a slot is arranged, into which a part of the other part can be introduced.

9. The implant as claimed in claim 1, wherein both parts are arranged behind one another in a sagittal direction.

10. The implant as claimed in claim 1, wherein both parts can be latched or locked on to one another in an end position detachably or undetachably.

11. The implant as claimed in claim 1, wherein both parts in each case have at least one shoulder, which rest on each other when both parts are joined in the end position.
